(19) United States
(12) Patent Application Publication
(10) Pub. No.: US 2011/0294852 A1
(43) Pub. Date: Dec. 1, 2011

(54) Pharmaceutical Formulation Comprising Metformin and Repaglinide

(75) Inventors: Bent Højgaard, Allerod (DK); Breian Knudsen, Albertslund (DK); Stella Rudkjær Rasmussen, Dyssegard (DK); Lene Orup Jacobsen, Gentofte (DK)

(73) Assignee: Novo Nordisk A/S, Bagsvaerd (DK)

(21) Appl. No.: 13/205,678

(22) Filed: Aug. 9, 2011

Related U.S. Application Data
(63) Continuation of application No. 12/442,432, filed on May 6, 2009, now abandoned.

(30) Foreign Application Priority Data
Sep. 29, 2006 (EP) 06121562.0

(51) Int. Cl. A61K 31/451; A61P 3/10

(52) U.S. Cl. 514/331

(57) ABSTRACT

The invention is related to a pharmaceutical composition comprising repaglinide in combination with metformin or a salt thereof in unit dosage form wherein a pre-formulation of the repaglinide has a pH independent dissolution profile and a relative humidity of less than about 25% prior to mixing with the metformin or a salt thereof; and optionally one or more pharmaceutically acceptable excipients and to a method for its preparation.

### CROSS-REFERENCE TO RELATED APPLICATIONS

This application is a Continuation of U.S. patent application Ser. No. 12/442,432, filed Mar. 23, 2009, which is a 35 U.S.C. §371 national stage application of International Patent Application PCT/EP2007/060343 (published as WO 2008/037807 A1), filed Sep. 28, 2007, which claimed priority of European Patent Application 06121562.0, filed Sep. 29, 2006.

### FIELD OF THE INVENTION

The present invention relates to a unit-dose combination formulation for the combined delivery of repaglinide and metformin or a salt thereof. Such a formulation can be used for the treatment of non-insulin dependent diabetes mellitus (NIDDM) and the improvement of glycemic control. The present invention also relates to processes for the preparation of such combination unit-dose formulations and the use of such combination formulations in the treatment of NIDDM.

### BACKGROUND OF THE INVENTION

Diabetes is characterised by an impaired glucose metabolism manifesting itself among other things by an elevated blood glucose level in the diabetic patients. Underlying defects lead to a classification of diabetes into two major groups: Type I diabetes, or insulin demanding diabetes mellitus (IDDM), which arises when patients lack β-cells producing insulin in their pancreatic glands, and type 2 diabetes, or non-insulin dependent diabetes mellitus (NIDDM), which occurs in patients with an impaired β-cell function besides a range of other abnormalities.

Type 1 diabetic patients are currently treated with insulin, while the majority of type 2 diabetic patients are treated either with sulphonylureas that stimulate β-cell function or with agents that enhance the tissue sensitivity of the patients towards insulin, or with insulin. Among the agents applied to enhance tissue sensitivity towards insulin, metformin is a representative example.

Repaglinide (PRANDIN® tablets or NOVONORM® tablets, Novo Nordisk) is a secretagogue and was introduced into the market for the treatment of NIDDM by helping the body to release more insulin. Unlike some other secretagogues, however, PRANDIN® is taken before meals to help control glucose levels after meals when these levels are most likely to spike.

The monograph for PRANDIN® tablets in the Physicians Desk Reference (PDR 2002, page 2433) describes the use of repaglinide alone or in combination with a sulfonylurea or biguanide such as metformin where a patient is poorly controlled on either of the compounds alone. The use of repaglinide along with metformin has been demonstrated to be synergistic in rats in controlling NIDDM related symptoms when compared with the use of either of the compounds alone.

WO 01/32158 describes the use of combinations of metformin with other antidiabetic agents such as sulfonylureas in treating NIDDM in patients. WO 98/56378 describe a novel regimen for the treatment of NIDDM in patients poorly controlled on metformin alone comprising administration of metformin and repaglinide together. US2003/0224046 relates to a unit dose combination composition of a short-acting oral hypoglycemic biologically active agent such as for example repaglinide or nateglinide and a long-acting oral hypoglycemic biologically active agent such as metformin.

A chemically and physically stable dosage form which can provide therapeutic levels of a metformin and repaglinide from the same unit-dose formulation in a fashion similar to each of the separate products available commercially would be extremely beneficial in clinical practice for glycemic control in the treatment of NIDDM for patients poorly controlled on either metformin or repaglinide alone.

### SUMMARY OF THE INVENTION

The present invention relates to a unit-dose combination formulation for the combined delivery of repaglinide, and metformin or a salt thereof. In a further aspect the present invention is directed to a unit dose formulation comprising metformin or a salt thereof and repaglinide in the form of a pre-formulation. Such a formulation can be used for the treatment of non-insulin dependent diabetes mellitus (NIDDM) and the improvement of glycemic control. The present invention also relates to processes for the preparation of such combination unit-dose formulations and the use of such combination formulations in the treatment of NIDDM.

In one embodiment the invention is related to a pharmaceutical composition comprising, repaglinide in combination with metformin or a salt thereof in unit dosage form wherein a pre-formulation of the repaglinide has a pH independent dissolution profile and a relative humidity of less than about 25% prior to mixing with the metformin or a salt thereof; and optionally one or more pharmaceutically acceptable excipients.

In one embodiment the repaglinide pre-formulation has a relative humidity of less than 20%.

In another embodiment the repaglinide pre-formulation has a relative humidity of 5-20%.

In another embodiment the repaglinide pre-formulation has a relative humidity of 7-17%.

In another embodiment the repaglinide pre-formulation has a relative humidity of 7-10%.

In one embodiment repaglinide preformation is obtained by spray drying and the metformin is in the form of a granulate.

In a further embodiment the invention is related to a method for the preparation of a pharmaceutical composition comprising repaglinide in combination with metformin in unit dosage form comprising (a) preparing a repaglinide pre-formulation granulate comprising repaglinide having a pH independent dissolution profile; (b) drying the repaglinide pre-formulation granulate to a relative humidity of less than about 25%; (c) mixing the repaglinide pre-formulation granulate from (b) with metformin or a salt thereof and optionally one or more pharmaceutically acceptable excipients; and (d) processing the mixture of (c) into the unit dosage form.

### DESCRIPTION OF THE DRAWINGS

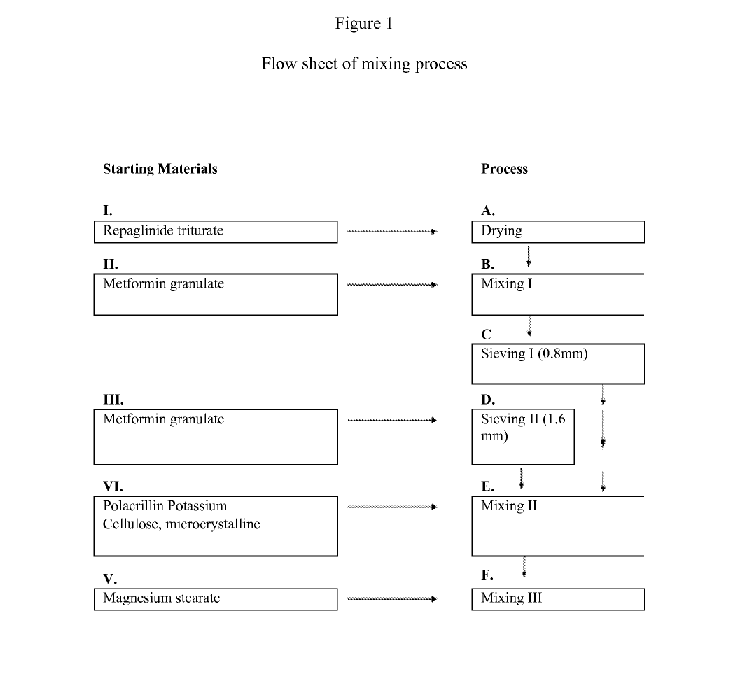

In the drawings FIG. 1 illustrates a flow sheet of the mixing process of the individual components.

### DESCRIPTION OF THE INVENTION

The present invention relates to a unit dosage form formulation comprising 1) a pre-formulation of repaglinide and at least one or more pharmaceutically acceptable excipients, which pre-formulation has a pH independent dissolution profile and a relative humidity of less than about 25% prior to mixing with the metformin, 2) metformin or a salt thereof and 3) optionally one or more pharmaceutically acceptable excipients.

The term “pre-formulation”, “repaglinide pre-formulation” or “pre-formulation of repaglinide” as used herein is defined as a formulation of repaglinide and at least one or more pharmaceutically acceptable excipients.

The term “having a pH independent dissolution profile” when used herein to describe a formulation means that at least 60% of the drug in the formulation has been released within 15, min. when tested under mild dissolution test conditions (≦50 rpm/paddle) in different buffers having a pH 1.0-7.5. In a further aspect, the pH independent dissolution profile means that at least 65% of the drug in the formulation has been released within 15, min. when tested under mild dissolution test conditions (≦50 rpm/paddle) in different butlers having a pH 1.0-7.5. In yet a further aspect, at least 85% is released within 15. min. when tested under mild dissolution test conditions (≦50 rpm/paddle) in different buffers having a pH 1.0-5.0.

The term “mixing” is used in its normal meaning and will encompass any conventional mixing process including granulation.

In one aspect of the invention, the unit dosage form formulation releases each drug from the unit-dose formulation at a rate similar to or faster than that of either or both the individually marketed products in individual form (viz., repaglinide from formulations such as PRANDIN® tablets or NOVONORM® tablets (Novo Nordisk), and metformin from formulations such as GLYCOPHAGE® tablets (Merck)). In order to determine the tablets similarity the dissolution profile of the unit dosage form formulation according to the invention and the individual products may be compared as described in the similarity test (f2-test) (25 Aug. 1997. Dissolution Testing of Immediate Release Solid Oral Products. Guidance for industry).

In a further aspect of the invention, the unit dosage form formulation according to the invention is an immediate release formulation. The term “immediate release formulation” as used herein is defined as a formulation showing a release of the active substance(s) which is not deliberately modified by a special formulation design and/or manufacturing method. In one aspect of the unit dosage form formulation according to the invention 85% or more of the drug(s) is released within 15, min. when tested under mild dissolution test conditions (≦50 rpm/paddle) in 0.1N HCl.

In a further aspect of the invention, the unit dosage form formulation according to the invention is physically and chemically stable. Stability of the tablets can be measured at accelerated as well as at long term storage conditions for periods of several weeks. Experiments can be performed at different temperatures and humidities

In a further aspect of the invention, the unit dosage form formulation according to the invention is homogonous. A unit dosage form is “homogonous” as used herein when it has a uniform distribution of active ingredient(s) as described in e.g. “Guidance for Industry, Powder Blends and Finished Dosage Units—Stratified In-Process Dosage Unit Sampling and Assessment”, FDA, October 2003.

It is a particular challenge to a pharmaceutical scientist to develop the type of formulation as described herein where one active substance, the low-dose low-solubility repaglinide, is uniformly dispersed in the other active substance, the high-dose highly water-soluble metformin or a salt thereof, and where both active substances are released at the desired rate.

It has now been found that by using a pre-formulation of repaglinide having a pH independent dissolution profile for the preparation of the unit dosage form formulation according to the invention a homogeneous and stable product from which the active substances releases at the desired rate may be obtained.

It has further been found that the relative humidity of the pre-formulation of repaglinide has an influence on the homogeneity of the repaglinide in the final drug product. In one embodiment of the invention, the pre-formulation of repaglinide has a relative humidity of less than 25%. In a further embodiment, the pre-formulation of repaglinide has a relative humidity of less than 20%. In yet a further embodiment, the pre-formulation of repaglinide has a relative humidity of 5-20%. In a further embodiment, the pre-formulation of repaglinide has a relative humidity of 7-17%.

The term “relative humidity” as used herein is defined as the ratio of the water vapor density (mass per unit volume) to the saturation water vapor density, usually expressed in percent:

$$Relative Humidity ( RH ) = \frac{( Actual Vapor Density )}{( Saturation Vapor Density )} \times 100 \%$$

Relative humidity is also approximately the ratio of the actual to the saturation vapor pressure.

$$Relative Humidy ( RH ) = \frac{( Actual Vapor Pressure )}{( Saturation Vapor Pressure )} \times 100 \%$$

Metformin may be used in the faun of a salt such as e.g. hydrochloride, acetate, maleate, fumarate, succinate and other salts. A detailed description of the different salts of metformin is described in the literature and is available in U.S. Pat. No. 6,031,004, which is herein incorporated by reference in its entirety. In one aspect of the invention, metformin is metformin hydrochloride.

The metformin or metformin hydrochloride or other pharmaceutically acceptable salts of metformin are in one aspect of the invention present in an amount from about 30% to about 95% by weight, in a further aspect present in an amount from about 55% to about 90% by weight, and in yet a further aspect present in an amount from about 75% to about 85% by weight of the unit dose form.

In one aspect of the invention, the metformin or a salt thereof is present in the unit dose form in an amount of from 100 mg to 2000 mg.

In another aspect of the invention, the metformin or a salt thereof is present in the unit dose form in an amount of from 250 mg to 1000 mg.

In a further aspect of the invention, the metformin or a salt thereof is present in the unit dose form in an amount of from 500 mg to 1000 mg.

In a further aspect of the invention, the metformin or a salt thereof is present in the unit dose form in an amount of from 500 mg to 850 mg.

Repaglinide is (S)-(+)-2-ethoxy-4-[2-[[3-methyl-1-[2-(1-piperidinyl)phenyl]butyl]-amino]-2-oxo-ethyl]benzoic acid, a compound described La. in European patent application publication No. 0 589 874 (to Dr. Karl Thomae GmbH).

In one embodiment repaglinide is present in an amount of from about 0.05% to about 5.0% by weight of the unit dose form.

In one aspect of the invention, the repaglinide is present in the unit dose four in an amount of from 0.20 mg to 5.0 mg.

In a further aspect of the invention, the repaglinide is present in the unit dose form in an amount of 0.5 mg to 2.0 mg.

In another embodiment repaglinide is present in an amount of from about 0.1% to about 1.0% by weight of the unit dose form.

In a further aspect of the invention, the repaglinide is present in the nit dose form in an amount of 0.5 mg to 1.0 mg.

In a still further aspect of the invention, the repaglinide is present in the unit dose form in an amount of 1.0 mg to 2.0 mg.

In one embodiment of the invention will comprise a pharmaceutical composition of repaglinide in combination with metformin unit dosage form wherein then metformin is present in an amount of from 500 to 850 mg and repaglinide is present in an amount of from 1.0 to 2.0 mg in the unit dose form.

In a further embodiment of the invention will comprise a pharmaceutical composition of repaglinide in combination with metformin unit dosage form wherein then metformin is present in an amount of 850 mg and repaglinide is present in an amount of from 1.0 mg in the unit dose form.

In a further embodiment of the invention will comprise a pharmaceutical composition of repaglinide in combination with metformin unit dosage form wherein then metformin is present in an amount of 500 mg and repaglinide is present in an amount of from 2.0 mg in the unit dose form.

### Preparation of the Pre-Formulation of Repaglinide Having a pH Independent Dissolution Profile

In one aspect of the invention, the repaglinide pre-formulation comprises repaglinide and one or more pharmaceutically acceptable excipients selected from the group consisting of a solubiliser, a binder, a basic agent, a solvent and a filler.

The term “basic agent” represents basic agents commonly used in the formulation of pharmaceuticals. Suitable basic agents for preparation of the pre-formulation of repaglinide include a number of inorganic or organic bases which are physiologically harmless, that is, pharmaceutically acceptable, at least in the dosage ranges used, such as sodium hydroxide solution, potassium hydroxide solution, ammonia, tert. sodium phosphate, diethanolamine, ethylenediamine, N-methylglucamine, or L-lysine. The molar ratio of active substance to basic excipient or mixtures of excipients is preferably from about 1:1.1 to 1:10, but a greater excess of base may also be advantageous in some cases.

The term “solubiliser” represents solubilisers commonly used in the formulation of pharmaceuticals. Examples of such substances suitable for preparation of the pre-formulation of repaglinide include polyvinyl pyrrolidones, polyethylene glycol 4000 or 6000, polyethoxylated sorbitan mono-oleates, sobitol, polyoxyethylene polyoxypropylene polymers, glycerol polyethylene glycoloxy stearates, and polyoxyethylene fatty alcohol ethers. Both the nature of the solubilizing substance and also the proportions used are important in determining the dissolution rate of the active substance. The ratio of active substance, e.g., repaglinide, to the total quantity of solubilizing substances is from about 1:1 to 1:10 (by weight).

The term “binder” represents binders commonly used in the formulation of pharmaceuticals. Examples of such binders suitable for preparation of the pre-formulation of repaglinide are polyvinylpyrrolidone, copolyvidone (cross-linked polyvinylpyrrolidone), polyethylene glycol, sucrose, dextrose, corn syrup, polysaccharides (including acacia, tragacanth, and guar), gelatin, and cellulose derivatives (including hydroxypropyl methylcellulose, hydroxypropyl cellulose, and sodium carboxymethylcellulose).

In one aspect of the invention, the repaglinide pre-formulation is prepared by making a solution of the active substance, basic excipient(s), binder(s) and solubilizing substance(s) primarily using a solvent such as water or other polar solvents such as lower alcohols, e.g., ethanol, isopropanol, ketones such as acetone, or mixtures of these substances with water.

The solutions thus prepared are applied to fillers such as water-insoluble fillers. Substances suitable for this purpose are preferably those which enlarge the surface area such as highly dispersed silicon dioxide, microcrystalline cellulose (such as AVICEL), basic aluminum oxide, magnesium-aluminium-trisilicates, cross-linked polyvinyl pyrrolidone, sodium carboxymethyl starch, tricalcium phosphate, calcium biphosphate, and mixtures thereof. Generally a ratio of active substance to carrier of from about 1:1 to 1:12 parts by weight is sufficient. Particularly suitable carriers are those which do not dissolve in water or some other appropriate solvent; these carriers permit easier handling both in the incorporation of the active substance and also in the further processing of the intermediate product.

In one aspect of the invention, the repaglinide pre-formulation comprises repaglinide and microcrystalline cellulose.

In one aspect of the invention, the pharmaceutically acceptable excipients in the repaglinide pre-formulation are Poloxamer 188, Povidone K25, meglumine and purified water.

In one aspect of the invention, the pre-formulation of repaglinide is obtained by conventional spray drying.

The repaglinide pre-formulation is in a one embodiment obtained by the following method: repaglinide and one or more pharmaceutically acceptable excipients such as a solubilizer, a binder and a basic ingredient is mixed, and a solvent is added. The mixture is then sprayed into hot dry air, so that the liquid evaporates leaving granules. The granules are then mixed with a filler to obtain a triturate, and the Obtained mixture is screened.

In one aspect of the invention, the pre-formulation is a triturate. This triturate is subjected to a drying process to get a relative humidity of less than 25% before being mixed with the metformin component. The drying process may be conducted in any convenient way such by blowing dry air trough the triturate or be drying with solid desiccates such as KHCO₃.

### Preparation of a Metformin Component

In one aspect of the invention, the metformin or a salt thereof is added to the pharmaceutical formulation in the form of granules which may be prepared by wet granulation, by dry granulation, by direct compression or melt granulation.

A general method of manufacture involves in one aspect of the invention, blending of metformin or a salt thereof, with a binder and a water-soluble diluent. This blend is then granulated with a solvent and optionally a lubricant and milled, if necessary. The granules are dried and reduced to a suitable size.

In one aspect of the invention, the mean particle size of the granules is from 0.0075-1 mm. In a further aspect of the invention, the mean particle size of the granules is from 0.01-0.8 mm. In a further aspect of the invention, the mean particle size of the granules is from 0.1-0.6 mm.

In one aspect of the invention, the metformin or a salt thereof is in the form of a metformin granulate.

In a further aspect of the invention, the metformin granulate comprises one or more pharmaceutically acceptable excipients selected from the group consisting of a binder, a solvent, a water-soluble diluent and optionally a lubricant.

The term “water-soluble diluent” represents compounds typically used in the formulation of pharmaceuticals, such as sugars (including lactose, sucrose and dextrose), polysaccharides (including dextrates and maltodextrin), polyols (including mannitol, xylitol and sorbitol) and cyclodextrins.

In a further aspect of the invention, the metformin granulate comprises metformin hydrochloride and the pharmaceutically acceptable excipients are Povidone K25, purified water, sorbitol and Macrogol 6000.

### Preparation of a Unit Dosage Form

In one aspect of the invention, a method for the preparation of a unit dosage form comprising the following steps: preparing a repaglinide pre-formulation, mixing the repaglinide pre-formulation, metformin or a salt thereof optionally in the form of a granulate and optionally one or more pharmaceutically acceptable excipients, and compressing the mixture into tablets and optionally film-coating the obtained tablets, is provided. In one aspect of the invention, the repalinide pre-formulation is spray-dried before mixing thereof with the metformin or a salt thereof optionally in the form of a granulate and optionally one or more pharmaceutically acceptable excipients.

The pharmaceutically acceptable excipients to be used in the preparation of a unit-dose form can be chosen from those routinely used in the art of preparation of pharmaceutical solid dosage forms. Such excipients include, but are not limited to, binding agents, bulking agents, disintegrants, glidants, wetting agents, lubricating agents, pigments, dyes and the like and are known to persons skilled in the art of developing and manufacturing pharmaceutical solid oral dosage forms. The choice of the tablet shape and size can be chosen by a person skilled in the art of preparation of pharmaceutical solid oral dosage forms.

Examples of pharmaceutically excipients may for example be inert diluents, such as mannitol, maltodextrin, kaolin, calcium carbonate, sodium carbonate, lactose, calcium phosphate or sodium phosphate; granulating and disintegrating agents, for example corn starch; binding agents, for example, starch, gelatine, polymers or acacia; and lubricating agents, for example magnesium stearate, stearic acid or talc.

The term “disintegrant” represents compounds such as starches, clays, celluloses, gums, cross-linked polymers (such as cross-linked polyvinylpyrrolidone and cross-linked sodium carboxymethylcellulose, sodium starch glycolate, low-substituted hydroxypropyl cellulose, sodium bicarbonate, polacrillin potassium, and soy polysaccharides. Preferably, the disintegrant is polacrillin potassium.

The term “lubricant” represents compounds frequently used as lubricants or glidants in the preparation of pharmaceuticals, such as talc, magnesium stearate, calcium stearate, stearic acid, colloidal silicon dioxide, magnesium carbonate, magnesium oxide, calcium silicate, microcrystalline cellulose, starches, mineral oil, waxes, glyceryl behenate, polyethylene glycol, sodium benzoate, sodium acetate, sodium chloride, sodium laurylsulfate, sodium stearyl fumarate, and hydrogenated vegetable oils. Preferably, the lubricant is magnesium stearate or talc.

The combination unit-dose tablet of the present invention can be prepared and coated by processes known to persons skilled in the art of manufacturing solid oral dosage forms. The optional coating, when present, is generally water-soluble and should dissolve rapidly when administered to the patient, preferably within 5 minutes of ingestion.

The combination unit-dose formulations of the present invention preferably contain from about 0.25 mg to about 5 mg of repaglinide and from about 200 mg to about 1200 mg of metformin or a salt thereof. More preferably, the combination unit-dose formulations contain from about 0.5 mg to about 2.0 mg of repaglinide and from about 500 mg to about 1000 mg of metformin or a salt thereof.

In addition, the formulations of the present invention show a dissolution profile substantially similar to that of the individually marketed products.

The dissolution profile of the unit dose formulation according to the invention may be measured in simulated gastric juice and in several aqueous buffer solutions in the pH range of 3-7.5. The paddle method may be used (apparatus 2 in USP, apparatus 1 in Ph.Eur.) Dissolution of repaglinide is strongly pH-dependant. The pH-value of 5.0 is the most sensitive level for performing dissolution tests. At this pH-value change in e.g. the spray dried granules inside the tablet can be clearly detected because of its specific solubility.

In one aspect of the invention, the unit dose formulation comprises repaglinide pre-formulation, metformin granulate, a filler, a disintegrant and a lubricant.

In another aspect of the invention, a method of treatment or prevention of hyperglycemia, type 2 diabetes, impaired glucose tolerance, type 1 diabetes, obesity, hypertension, syndrome X, dyslipidemia, cognitive disorders, atheroschlerosis, myocardial infarction, coronary heart disease and other cardiovascular disorders, stroke, inflammatory bowel syndrome, dyspepsia and gastric Ulcers in a patient in need of such a treatment or prevention, comprising administering to the patient a unit dosage form formulation according to the invention, is provided.

In another aspect of the invention, a method for delaying or preventing disease progression in type 2 diabetes in a patient in need of such a treatment, comprising administering to the patient a unit dosage form formulation according to the invention, is provided.

In another aspect of the invention, a method for decreasing food intake, decreasing β-cell apoptosis, increasing β-cell function and β-cell mass, and/or for restoring glucose sensitivity to β-cells in a patient in need of such treatment, comprising administering to the patient unit dosage form formulation according to the invention, is provided.

The treatment with the unit dosage form formulation according to the present invention may also be combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity.

In one embodiment, the unit dosage form formulation according to the invention is used for the preparation of a medicament for the treatment or prevention of hyperglycemia, type 2 diabetes, impaired glucose tolerance, type 1 diabetes, obesity, hypertension, syndrome X, dyslipidemia, cognitive disorders, atheroschlerosis, myocardial infarction, stroke, coronary heart disease and other cardiovascular disorders, inflammatory bowel syndrome, dyspepsia and gastric ulcers.

In another embodiment, a unit dosage form formulation according to the invention is used for the preparation of a medicament for delaying or preventing disease progression in type 2 diabetes.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term “prevention of a disease” as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated. The citation and incorporation of patent documents herein is done for convenience only and does not reflect any view of the validity, patentability and/or enforceability of such patent documents. The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a formulation described herein as comprising a particular element should be understood as also describing a formulation consisting of that element, unless otherwise stated or clearly contradicted by context).

### EXAMPLES

### Example 1

TABLE 1
Unit dose Composition for Repaglinide
1.0 mg and Metformin 850 mg Tablets

| | Ingredient | | Quantity in mg |
|---|---|---|---|
| Repaglinide | | | |
| Triturate | | | |
| 1 | Repaglinide | Active | 1.00 |
| 2 | Poloxamer 188 | Solubiliser | 0.286 |
| 3 | Povidone K25 | Binder | 0.286 |
| 4 | Meglumine | Basic Agent | 0.500 |
| 5 | Cellulose, | Filler | 12.0 |
| | Microcrystalline | | |
| Metformin | | | |
| granulate | | | |
| 6 | Metformin | Active | 850 |
| | Hydrochloride | | |
| 7 | Povidone K25 | Binder | 34.0 |
| 8 | Sorbitol | Water-soluble diluen | 17.0 |
| 9 | Macrogol 6000 | Lubricant | 8.50 |
| Excipients | | | |
| 10 | Cellulose, | Filler | 65.6 |
| | Microcrystalline | | |
| 11 | Polacrillin | Disintegrant | 30.8 |
| | Potassium | | |
| 12 | Magnesium | Lubricant | 5.13 |
| | Stearate | | |
| 13 | Opadry | Filmcoating | 24.1 |
| | | formulation | |

The tablet with the composition formula listed in table 1 is prepared as described in the method of example 3.

### Example 2

TABLE 2
Unit_dose Composition for Repaglinide
2.0 mg and Metformin 500 mg Tablets

| | Ingredient | | Quantity mg |
|---|---|---|---|
| Repaglinade | | | |
| Triturate | | | |
| 1 | Repaglinide | Active | 2.00 |
| 2 | Poloxamer 188 | Solubiliser | 0.572 |
| 3 | Povidone K25 | Binder | 0.572 |
| 4 | Meglumine | Basic Agent | 1.00 |
| 5 | Cellulose, | Filler | 24.0 |
| | Microcrystalline | | |
| Metformin | | | |
| granulate | | | |
| 6 | Metformin | Active | 500 |
| | Hydrochloride | | |
| 7 | Povidone K25 | Binder | 20.0 |
| 8 | Sorbitol | Water-soluble | 10.0 |
| | | diluen | |
| 9 | Macrogol 6000 | Lubricant | 5.00 |
| Excipients | | | |
| 10 | Cellulose, | Filler | 40.0 |
| | Microcrystalline | | |
| 11 | Polacrillin | Disintegrant | 18.8 |
| | Potassium | | |
| 12 | Magnesium | Lubricant | 3.13 |
| | Stearate | | |
| 13 | Opadry | Filmcoating | 14.7 |
| | | formulation | |

The tablet with the composition formula listed in table 2 is prepared as described in the method of example 3.

### Example 3

Repaglinide, Poloxamer 188, Povidone K25 and Meglumine is dissolved in purified water and spray-dried. The spray-dried powder is mixed with Cellulose, Microcrystalline to form a triturate in a suitable mixer.

Metformin Hydrochloride, Povidone K25, Sorbitol and Macrogol 6000 is granulated with Purified water in a suitable high shear mixer and dried.

The Repaglinide triturate and Metformin granulate was mixed with Cellulose, Microcrystalline and Polacrillin Potassium in a number of mixing steps and suitable mixers. The blend was lubricated with Magnesium, Stearate and compressed to into tablets on a tablets press and filmcoated.

One embodiment of the mixing process of the two active components is illustrated in FIG. 1 and will be explained further below.

### Drying Process, (Process A)

The water activity in the repaglinide triturate is critical to avoid electrostatic behaviour and segregation. The repaglinide triturate is dispensed in bags (units). Desiccant bags are randomly distributed to every unit. The bags are carefully sealed and placed in sealed drums. The water activity is measured regularly in each unit until the correct water activity is achieved (water activity ≦12%) but not longer than 30 days. The final determination of water activity is conducted on each unit directly before mixing. Water activity in all units must be equal to or below the desired value

### Mixing and Sieving (Process B-D)

The desiccant bags are removed from the repaglinide triturate. A part of the metformin HCl granulate and the repaglinide triturate is mixed in a diffusive double cone mixer. The combined pre-blend is sieved in a rotating impeller 0.8 mm directly into a double cone mixer. Sieving is conducted to eliminate lumps in the pre-blend and secure a uniformly particle size distribution.

### Sieving and Mixing (Process E-F)

The remaining part of metformin HCl granulate is sieved in a rotating impeller (1.6 mm) into the double cone mixer. The sieved metformin HCl granulate, the sieved pre-blend, polacrillin potassium and cellulose microcrystalline is mixed in a diffusive double cone mixer. Finally magnesium stearate is added and the last step of the mixing is performed.

1. A pharmaceutical composition comprising repaglinide in combination with metformin or a salt thereof in unit dosage form wherein a pre-formulation of the repaglinide has a pH independent dissolution profile and a relative humidity of less than about 25% prior to mixing with the metformin or a salt thereof; and optionally one or more pharmaceutically acceptable excipients.

2. A pharmaceutical composition according to claim 1, wherein the repaglinide pre-formulation has a relative humidity of less than 20%.

3. A pharmaceutical composition according to claim 2, wherein the repaglinide pre-formulation has a relative humidity of 5-20%.

4. A pharmaceutical composition according to claim 3, wherein the repaglinide pre-formulation has a relative humidity of 7-17%.

5. A pharmaceutical composition according to claim 4, wherein the repaglinide pre-formulation has a relative humidity of 7-10%.

6. A pharmaceutical composition according to claim 1, wherein the repaglinide pre-formulation comprises one or more pharmaceutically acceptable excipients selected from the group consisting of a solubiliser, a binder, a basic agent, a solvent and a filler.

7. A pharmaceutical composition according to claim 6, wherein the pharmaceutically acceptable excipients in the repaglinide pre-formulation are Poloxamer 188, Povidone K25, meglumine and purified water.

8. A pharmaceutical composition according to claim 1, wherein the metformin or a salt thereof is in the form of a metformin granulate.

9. A pharmaceutical composition according to claim 1, wherein the metformin is metformin hydrochloride.

10. A pharmaceutical composition according to claim 8, wherein the metformin granulate further comprises one or more pharmaceutically acceptable excipients selected from the group consisting of a binder, a solvent, a water-soluble diluent and optionally a lubricant.

11. A pharmaceutical composition according to claim 10, wherein the pharmaceutically acceptable excipients in the metformin granulate are Povidone K25, purified water, sorbitol and Macrogol 6000.

12. A pharmaceutical composition according to claim 1, which comprises repaglinide pre-formulation, and metformin granulate, and optionally a filler, a disintegrant and a lubricant.

13. A pharmaceutical composition according to claim 1, wherein metformin or a salt thereof is present in an amount of from 250 mg to 1000 mg.

14. A pharmaceutical composition according to claim 1, wherein repaglinide is present in an amount of 0.5 mg to 2.0 mg.

15. A method for the preparation of a pharmaceutical composition comprising repaglinide in combination with metformin in unit dosage form comprising (a) preparing a repaglinide pre-formulation granulate comprising repaglinide having a pH independent dissolution profile; (b) drying the repaglinide pre-formulation granulate to a relative humidity of less than about 25%; (c) mixing the repaglinide pre-formulation granulate from (b) with metformin or a salt thereof, and optionally one or more pharmaceutically acceptable excipients; and (d) processing the mixture of (c) into the unit dosage form.
